(19) United States
(12) Patent Application Publication
(10) Pub. No.: US 2026/0175021 A1
(43) Pub. Date: Jun. 25, 2026

(54) CRYO-DELIVERY APPARATUS

(71) Applicant: TCEB LIMITED, Cambridge (GB)

(72) Inventor: Andrew GRACE, Cambridge (GB)

(21) Appl. No.: 19/126,381

(22) PCT Filed: Oct. 31, 2023
(86) PCT No.: PCT/IB2023/060960

§ 371 Date: May 1, 2025

Related U.S. Application Data
(60) Provisional application No. 63/422,092, filed on Nov. 3, 2022.

(51) Int. Cl. A61N 1/05; A61B 18/00; A61B 18/02; A61B 18/18

(52) U.S. Cl. CPC A61N 1/056; A61B 18/18; A61N 1/057; A61B 2018/00041; A61B 2018/0212; A61B 2018/0262; A61B 2018/0293

(57) ABSTRACT

In accordance with the inventive concepts, provided is a cryo-delivery apparatus and method. In various embodiments, the cryo-delivery apparatus is useful to perform a percutaneous cryo-delivery method. In one embodiment, a therapeutic is adhered by freezing to a distal end of a catheter, the distal end is brought into contact with tissue to be treated, e.g., directed percutaneously, and the distal end is then thawed to deliver the therapeutic to the tissue. In another embodiment, the distal end is directed to the tissue to be treated, e.g., percutaneously, then brought to freezing to established stable coupling to the tissue, a therapeutic is then directed through the catheter and distal end to the treatment site, the distal end is thawed to release and deliver the therapeutic to the tissue, and the catheter can then be withdrawn.

### CROSS-REFERENCE TO RELATED APPLICATION

This patent application claims the benefits of priority under 35 U.S.C. § 119 to U.S. Provisional Ser. No. 63/422,092, filed Nov. 3, 2022, the entirety of which is incorporated herein by reference.

### FIELD OF INTEREST

The present inventive concepts relate to the field of medicine and, in particular, to methods and apparatuses for delivering a therapeutic within a body, e.g., percutaneously.

### BACKGROUND

There are many situations where the delivery of medications within the body offers an approach to effective treatment of a medical condition. For example, cardiac disease is a progressive and fatal disease affecting about 64 million people globally; and its prevalence is increasing. Treatments for heart disease can require delivery of a treatment agent or medication to a target treatment site within the body, or within the heart itself. To effect delivery, different types of administration routes can be considered with respect to the heart, such as minimally invasive surgery, coronary surgery, and catheter. Each administration route has its own pros and cons. The simplest of these procedures, allowing the quickest recovery, is the catheter. But a catheter is not always a possibility depending on cardiac condition and the medication to be delivered. Additionally, the dynamic nature of a beating heart can present significant challenges, since stable contact with the heart wall is challenging.

Fifty percent (50%) of patients that have experienced heart failure have a 5-year mortality rate. This is because heart failure causes significant cell damage, as seen in FIG. 1. In the infarcted area, scar tissue can form, resulting in a loss of cardiomyocytes, as shown in FIG. 2. Infarction is tissue death, or necrosis, caused by inadequate blood supply to the affected tissue. This damage compromises heart function, which can reduce quality of life and put the person at risk of a fatal cardiac incident. Effective treatment to address the scar tissue requires cell therapy and/or cell regeneration.

Certain medications, such as some biologics, can be used to effectively repair and/or regenerate damaged cells in the heart, such as on or within the heart walls. For example, biologics can include medications developed from blood, proteins, viruses, or living organisms, which can be used to prevent, treat, and cure many health conditions. To be effective, the cell treatment medication must be delivered to the site of the damaged heart tissue.

Intravenous, intracoronary, and intramyocardial are low retention approaches to delivering therapeutic biologics. Referring to FIG. 3, primary theories of low cell retention of the therapeutic in the damaged area are provided. The intravenous approach (a) suffers from poor homing, or low concentration of the therapeutic biologic delivered to the site of the cell damage. The intracoronary approach (b) can result in plugging and wash out. And the intramyocardial approach (c) can result in cell death and leakage. None of these approaches would provide high efficacy with respect to treating the damaged cell tissue. Potential viral delivery approaches could be considered, but these could have serious potential risks, such as possible off-target genotoxicity.

Approaches that are likely to provide higher concentrations of cell repair and regeneration therapeutics to the damaged cell site include cell sheets, cardiac patches, and intrapericardial cavity injections, as shown in FIG. 4. But only surgical approaches for delivery currently exist.

It would be advantageous to have a device and a method that delivers a cell repair and/or regenerative therapeutic to a site within the body of damaged cardiac cells. It would also be advantageous to apply such device and method for delivery of other types of therapeutics within the heart or chamber of the heart, within other organs and/or tissue, and/or at other sites within the body. It would be further advantageous for the device to include or be delivered by a catheter, such that the procedure is relatively simple, risks to the patient are minimized, and patient recovery and discharge are comparatively quick. These and other advantages of the inventive concepts will be apparent from this disclosure.

### SUMMARY

In accordance with the inventive concepts, provided is a cryo-delivery apparatus and method. In various embodiments, the cryo-delivery apparatus is useful to perform a percutaneous cryo-delivery method. In one embodiment, a therapeutic is adhered by freezing to a distal end of a catheter, the distal end is brought into contact with tissue to be treated, e.g., directed percutaneously, and the distal end is then thawed to deliver the therapeutic to the tissue. In another embodiment, the distal end is directed to the tissue to be treated, e.g., percutaneously, then brought to freezing to established stable coupling to the tissue, a therapeutic is then directed through the catheter and distal end to the treatment site, the distal end is thawed to release and deliver the therapeutic to the tissue, and the catheter can then be withdrawn.

According to an aspect of the inventive concepts, provided is a percutaneous cryo-delivery method, comprising: providing a steerable catheter having a probe tip having a probe tip temperature that is controllable by a thermal controller; adhering a therapeutic to the probe tip outside the body by using the thermal controller to maintain the probe tip temperature at or below freezing while in contact with the therapeutic; using the steerable catheter, directing the probe tip with the adhered therapeutic to a treatment site within the body while maintaining the probe tip at or below freezing; and using the thermal controller, delivering the therapeutic to the treatment site by causing the probe tip temperature to rise above freezing while in contact with the treatment site.

In some embodiments, the catheter defines at least one lumen and the probe tip defines at least one opening terminating the at least one lumen and the method includes delivering at least one therapeutic to the treatment site via the at least one lumen and the at least one opening.

According to another aspect of the inventive concepts, provided is a percutaneous cryo-delivery method, comprising: providing a steerable catheter defining at least one lumen and having a probe tip having at least one opening, where a probe tip temperature is controllable by a thermal controller; delivering the probe tip to contact a treatment site within the body; using the thermal controller, adhering the probe tip to tissue at the treatment site by bringing the probe tip temperature to or below freezing; and while maintaining the probe tip frozen adherence to the tissue, delivering a therapeutic to the tissue through the at least one lumen and the at least one probe tip opening.

According to another aspect of the inventive concepts, provided is a percutaneous cryo-delivery method, comprising: providing a steerable catheter defining a lumen and having a probe tip including a rim, where a rim temperature is controllable by a thermal controller; delivering the rim to contact tissue at a treatment site within the body; using the thermal controller, adhering the rim to the tissue by bringing the rim temperature to or below freezing; and while maintaining the rim in frozen adherence with the tissue, delivering a therapeutic to the tissue through the lumen and via the rim.

In some embodiments, the therapeutic is in the form of a material and/or at least one device.

In some embodiments, the therapeutic is in the form therapeutic a drug, biologic, material, and/or device having a treatment and/or ameliorative effect, which is delivered via the cryo-delivery catheter to a site and/or tissue within the body.

In some embodiments, the therapeutic includes a monitoring device, a pacing device, and/or a leadless pacing device.

In some embodiments, the therapeutic is a biologic.

In some embodiments, the therapeutic includes plastic bioelectronics.

In some embodiments, the therapeutic includes one or more of the following: RNA, DNA, stem cells, progenitor cells, tissue, drugs, and/or CRISPR-Cas.

In some embodiments, the therapeutic is a delivered in a format of a patch or sheet.

In some embodiments, the therapeutic is in the form of a gel, paste, liquid, solution, solid, or combinations thereof.

In some embodiments, the probe tip includes at least one opening and the method includes delivering at least one therapeutic through the at least one opening.

In some embodiments, the probe tip includes a plurality of openings and the method includes delivering a single therapeutic through the plurality of openings.

In some embodiments, the probe tip includes a plurality of openings and the method includes delivering a plurality of therapeutics through the plurality of openings.

In some embodiments, the probe tip includes at least one thermal insulator insulating the at least one opening and/or rim from the lumen or plurality of lumens and the therapeutic or plurality of therapeutics.

In some embodiments, the probe includes an electronic device including at least one electrode, such as a biopotential electrode and/or an ultrasound electrode.

In some embodiments, the at least one electrode can comprise at least one ring electrode.

In some embodiments, delivering the therapeutic includes delivering a monitoring device, a pacing device, and/or a leadless pacing device to the treatment site.

In some embodiments, the method further comprises further comprising using electroporation or electropermeabilization to increase a permeability of the tissue at the treatment site.

According to another aspect of the inventive concepts, provided is a cryo-delivery catheter, comprising: an elongate shaft defining at least one lumen; a probe tip coupled to a distal end of the shaft and having at least one opening terminating the at least one lumen; and a thermal controller configured to control a temperature of the probe tip to bring a probe tip temperature to or below freezing.

In some embodiments, the at least one opening includes at least one rim configured to achieve the probe tip temperature at or below freezing and configured to couple by freezing to tissue at a treatment site.

In some embodiments, the probe tip includes one opening defined by one rim.

In some embodiments, the probe tip includes a plurality of openings within one rim.

In some embodiments, the probe tip includes a plurality of openings defined by a plurality of rims.

In some embodiments, one or more of the plurality of rims can be independently thermally controlled to reach a temperature at or below freezing.

In some embodiments, the probe tip includes at least one insulator insulating the at least one opening, the at least one lumen, and/or the rim or plurality of rims.

In some embodiments, the probe tip includes one opening or rim and an insulator insulating the one opening or rim.

In some embodiments, the catheter further comprises at least one microtube configured to carry a refrigerant to at least one void in the probe tip and at least one conduit configured to evacuate refrigerant gas from the at least one void.

In some embodiments, the at least one lumen is configured as a pathway for at least one therapeutic to exit the probe tip via the at least one opening, rim, or plurality of rims while the at least one opening is frozen to the tissue at the treatment site.

In some embodiments, the probe tip further includes a temperature controllable end face the closes at least a portion of the probe tip and is configured to maintain a therapeutic by freezing.

In some embodiments, the probe tip end face is temperature controllable to transition above freezing to deliver the therapeutic to the tissue at the treatment site.

In some embodiments, the probe includes an electronic device including at least one electrode, such as a biopotential electrode and/or an ultrasound electrode.

In some embodiments, the at least one electrode can comprise at least one ring electrode.

In some embodiments, the catheter is configured to deliver a therapeutic in the form of a material and/or at least one device.

In some embodiments, the catheter is configured to deliver a therapeutic to a site and/or tissue within the body in the form of a drug, biologic, material, and/or device having a treatment and/or ameliorative effect.

In some embodiments, the catheter is configured to deliver a therapeutic to a site and/or tissue within the body that includes a monitoring device, a pacing device, and/or a leadless pacing device.

In some embodiments, the catheter is configured to deliver a therapeutic to a site and/or tissue within the body that includes a biologic.

In some embodiments, the catheter is configured to deliver a therapeutic to a site and/or tissue within the body that includes plastic bioelectronics.

According to another aspect of the inventive concepts, provided is a cryo-delivery probe: configured to couple to a distal end of a catheter shaft having at least one lumen; at least one opening configured to terminate the at least one lumen; and a temperature controllable tip configured to transition to a probe tip temperature at or below freezing to couple by freezing to tissue of a treatment site.

In some embodiments, the at least one opening includes at least one rim configured to achieve the probe tip temperature at or below freezing and configured to couple by freezing to tissue at a treatment site.

In some embodiments, the probe tip includes one opening defined by one rim.

In some embodiments, the probe tip includes a plurality of openings within one rim.

In some embodiments, the probe tip includes a plurality of openings defined by a plurality of rims.

In some embodiments, one or more of the plurality of rims can be independently thermally controlled to reach a temperature at or below freezing.

In some embodiments, the probe tip includes at least one insulator insulating the at least one opening, the at least one lumen, and/or the rim or plurality of rims.

In some embodiments, the probe tip includes one opening or rim and an insulator insulating the one opening or rim.

In some embodiments, the probe tip further includes a temperature controllable end face the closes at least a portion of the probe tip and is configured to maintain a therapeutic by freezing.

In some embodiments, the probe tip end face is temperature controllable to transition above freezing to deliver the therapeutic to the tissue at the treatment site.

In some embodiments, the probe tip includes an electronic device including at least one electrode, such as a biopotential electrode and/or an ultrasound electrode.

In some embodiments, the at least one electrode can comprise at least one ring electrode.

In some embodiments, the probe tip is configured to deliver a therapeutic in the form of a material and/or at least one device.

In some embodiments, the probe tip is configured to deliver a therapeutic to a site and/or tissue within the body in the form of a drug, biologic, material, and/or device having a treatment and/or ameliorative effect.

In some embodiments, the probe tip is configured to deliver a therapeutic to a site and/or tissue within the body that includes a monitoring device, a pacing device, and/or a leadless pacing device.

In some embodiments, the probe tip is configured to deliver a therapeutic to a site and/or tissue within the body that includes a biologic.

In some embodiments, the probe tip is configured to deliver a therapeutic to a site and/or tissue within the body that includes plastic bioelectronics.

### BRIEF DESCRIPTION OF THE DRAWINGS

The present inventive concepts will become more apparent in view of the attached drawings and accompanying detailed description. The embodiments depicted therein are provided by way of example, not by way of limitation, wherein like reference numerals refer to the same or similar elements. The drawings are not necessarily to scale, emphasis instead being placed upon illustrating aspects of the inventive concepts.

FIG. 1 is a diagram depicting heart failure progression, in accordance with the prior art;

FIG. 2 is a representation of a heart showing the formation of scar tissue and a loss of cardiomyocytes, in accordance with the prior art;

FIG. 3 is a diagram depicting different low cardiac cell retention theories, in accordance with the prior art;

FIG. 4 is a diagram depicting different low cardiac cell retention theories, in accordance with the prior art;

FIG. 5 is an example embodiment of cryo-delivery system using a heart as an example, in accordance with aspects of the inventive concepts;

FIG. 6A is an example embodiment of a cryo-deliver catheter distal end, in in accordance with aspects of the inventive concepts;

FIG. 6B is another example embodiment of a cryo-deliver catheter distal end, in in accordance with aspects of the inventive concepts;

FIG. 7 is an example embodiment of a cryo-delivery catheter, in accordance with aspects of the inventive concepts;

FIG. 8 is an embodiment of a cardiac sheet or patch adhered by freezing to the probe tip, in accordance with aspects of the inventive concepts;

FIG. 9 is an embodiment of a therapeutic material adhered by freezing to the probe tip, in accordance with aspects of the inventive concepts;

FIG. 10 is an embodiment of a therapeutic material including an electronic device adhered by freezing to the probe tip, in accordance with aspects of the inventive concepts;

FIG. 11A is an example embodiment of distal end of the cryo-delivery catheter of FIG. 6A, in accordance with aspects of the inventive concepts;

FIG. 11B is an example embodiment of distal end of the cryo-delivery catheter of FIG. 11A, in accordance with aspects of the inventive concepts;

FIG. 12(a)-(e) are example embodiments of different configurations of a distal end of a cryo-delivery catheter, in accordance with aspects of the inventive concepts;

FIG. 13 is a flowchart depicting an embodiment of a method of percutaneous delivery of a treatment agent, in accordance with aspects of the inventive concepts; and

FIG. 14 is a flowchart depicting another embodiment of a method of percutaneous delivery of a treatment agent, in accordance with aspects of the inventive concepts.

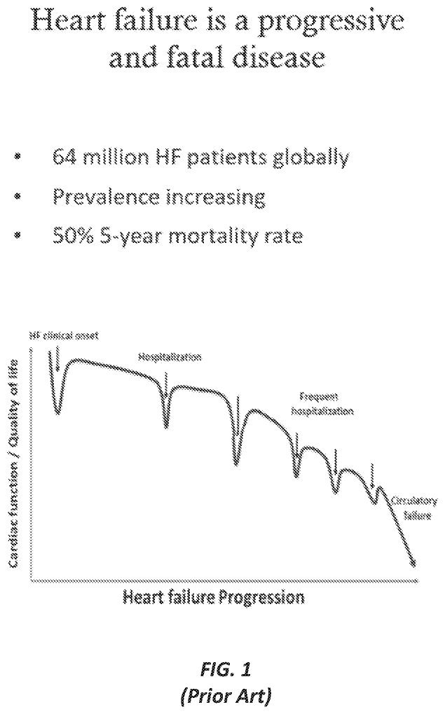

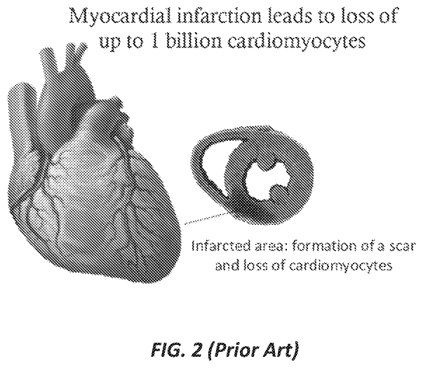

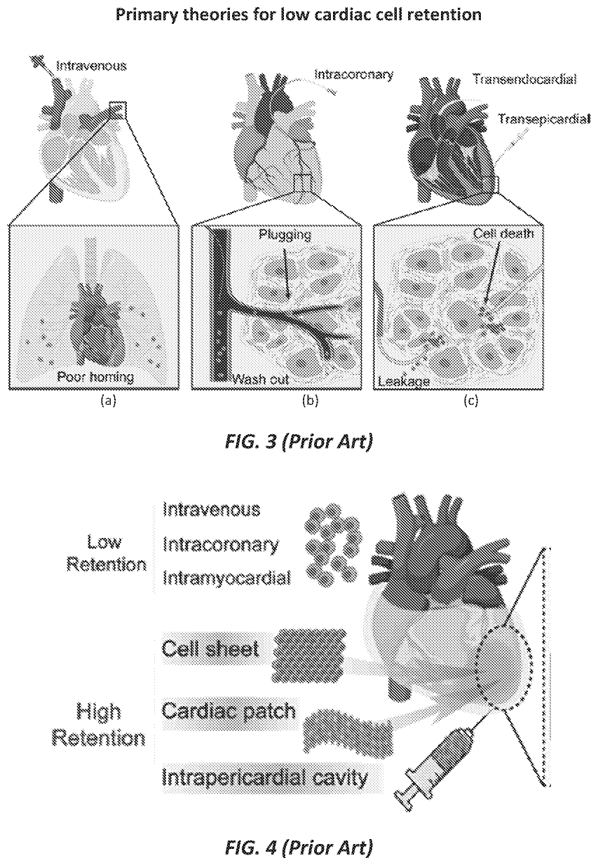

### DETAILED DESCRIPTION OF PREFERRED EMBODIMENTS

Various aspects of the inventive concepts will be described more fully hereinafter with reference to the accompanying drawings, in which some exemplary embodiments are shown. The present inventive concept may, however, be embodied in many different forms and should not be construed as limited to the exemplary embodiments set forth herein.

It will be understood that, although the terms first, second, etc. are be used herein to describe various elements, these elements should not be limited by these terms. These terms are used to distinguish one element from another, but not to imply a required sequence of elements. For example, a first element can be termed a second element, and, similarly, a second element can be termed a first element, without departing from the scope of the present invention. As used herein, the term “and/or” includes any and all combinations of one or more of the associated listed items.

It will be understood that when an element is referred to as being “on” or “connected” or “coupled” to another element, it can be directly on or connected or coupled to the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, steps, operations, elements, and/or components, but do not preclude the presence or addition of one or more other features, steps, operations, elements, components, and/or groups thereof.

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in the figures is turned over, elements described as “below” and/or “beneath” other elements or features would then be oriented “above” the other elements or features. The device may be otherwise oriented (e.g., rotated 90 degrees or at other orientations) and the spatially relative descriptors used herein interpreted accordingly.

Exemplary embodiments are described herein with reference to cross-sectional illustrations that are schematic illustrations of idealized exemplary embodiments (and intermediate structures). As such, variations from the shapes of the illustrations as a result, for example, of manufacturing techniques and/or tolerances, are to be expected. Thus, exemplary embodiments should not be construed as limited to the particular shapes illustrated herein, but are to include deviations in shapes that result, for example, from manufacturing or reasonably foreseeable alternatives.

To the extent that functional features, operations, and/or steps are described herein, or otherwise understood to be included within various embodiments of the inventive concept, such functional features, operations, and/or steps can be embodied in functional blocks, units, modules, operations and/or methods. And to the extent that such functional blocks, units, modules, operations and/or methods include computer program code, such computer program code can be stored in a computer readable medium, e.g., such as non-transitory memory and media, that is executable by at least one computer processor.

In accordance with the inventive concepts, provided is a cryo-delivery apparatus and method. In various embodiments, a cryo-delivery apparatus is configured and useful for delivering a therapeutic, e.g., in the form of a material and/or at least one device, to organs or other tissue within a body. In various embodiments, a therapeutic can be a drug, biologic, material, and/or device having a treatment and/or ameliorative effect, which is delivered via the cryo-delivery catheter to a site and/or tissue within the body. In various embodiments, such a device could be or include at least one wireless cardiac monitoring and/or pacing device. Such pacing devices could take the form, for example, of leadless pacing devices used with multisite pacing. The stability afforded by the cryo-delivery apparatus facilitates accurate, stable delivery in any selected endocardial or epicardial location. In various embodiments, using the cryo-delivery apparatus one can place biological/electronic/other material on the outer surface (epicardium) of the heart, in addition to the endocardium otherwise referred to herein, and monitor the same via electrophysiological and imaging modalities.

In some embodiments, the therapeutic can be adhered to a distal end or tip of the catheter by freezing before the catheter is directed through the body via a catheter sheath to a target site having tissue to be treated. Once the distal end with the therapeutic frozen thereto is brought into direct contact with the tissue, the distal end can be transitioned to a temperature above freezing to thaw and deliver the therapeutic to the tissue. This can be considered a method of direct delivery.

In other embodiments, a distal end of the catheter can be directed through the sheath to the target site where the tissue is located and, once the distal end is in contact with the tissue, the distal end can be brought to a temperature at or below freezing to create a frozen coupling between the distal end and the tissue. While the coupling is maintained, one or more therapeutics, e.g., in the form of a material and/or at least one device, can be delivered through the catheter lumen to the tissue via the distal end, which distal end can define one or more openings for delivering the therapeutic. Here, the therapeutic is tunneled through the catheter. This can be referred to as indirect delivery.

In some embodiments, both methods of treatment can be used in combination, i.e., direct delivery with adherence and indirect delivery with tunneling.

In various embodiments, a cryo-delivery apparatus can include an intravenous probe having at least one distal end probe tip capable of reaching temperatures sufficient to freeze the probe tip of the catheter to the tissue of a body, such as tissue of an organ. The distal end of the probe can be selectively transitioned between temperatures at or below freezing temperature and temperatures above freezing. A cryo-delivery console can include a temperature (or thermal) controller configured to control a temperature of the distal end, or probe tip, of the catheter to transition to or below freezing and/or above freezing.

Differentiators of a cryo-delivery catheter in accordance with the inventive concepts include: (i) stability and (ii) delivery of frozen biological material. Biological material, e.g., as a therapeutic, to be delivered can include one or more of the following: RNA, DNA, stem cells, progenitor cells, tissue, drugs, and/or CRISPR-Cas. The format of the biologic to be delivered can include one or more of: all above, viral vectors, nanoparticles, cell sheet, cardiac patches, needle, tunnelling tools, minor surgical (suction blood first) etc. In various embodiments, delivery can be achieved to the endocardial, epicardial, ventricles, and/or atria. In accordance with aspects of the inventive concepts, provided are two general technical approaches: (i) frozen catheter tip (−10 to −190° C.) and (ii) frozen sheath carrier.

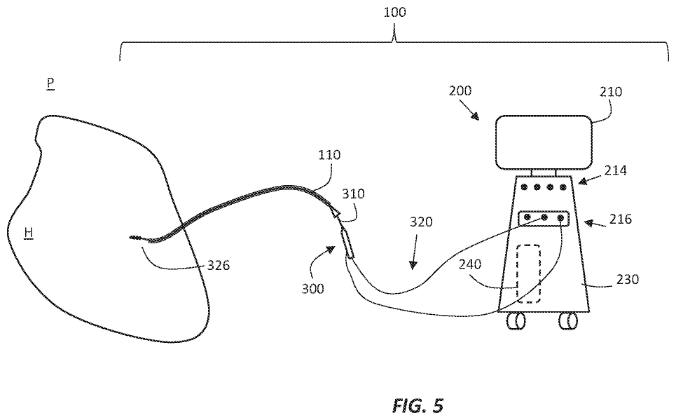

FIG. 5 is an example embodiment of cryogenic delivery (or “cryo-delivery”) system 100, in accordance with aspects of the inventive concepts. In FIG. 5, a heart H of a patient P is shown, and the cryo-delivery system 100 includes a console 200 and a cryo-delivery catheter 300. The cryo-delivery catheter 300 includes a cryogenic probe (or “cryoprobe”) 326 at a distal end of a catheter shaft 310.

The console 200 can include at least one display 210 and a processing unit 230 comprising one or more processors, memory, and power sources. The memory can store program logic executable by the one or more processors to perform one or more steps of cryo-delivery methods, such as direct and indirect delivery methods described herein. The processing unit 230 can include a thermal controller 240, which can include at least one of the one or more processors, configured to control a probe tip temperature. For example, the thermal controller 240 of the console 200 can be configured to control the flow of a refrigerant, such as liquid nitrogen, to the probe tip, thereby controlling the probe tip temperature at the distal end of the cryo-delivery catheter 300.

The cryo-delivery console 200 can include a console display 210 configured to display information, instructions, graphs, charts, anatomy, data streams, and/or real-time monitored parameters related to the patient P, the heart H, the cryo-delivery catheter 300 and/or its probe tip, and/or a procedure being performed. The console 200 can also include one or more user-interactive mechanisms 214, e.g., knobs, dials, switches, keys, keyboard, camera, and/or sensors, useful and/or necessary for controlling the console display and/or the cryo-delivery catheter 300, including the thermal controller 240. In some embodiments, the console display 210 can be a touch-sensitive display configured to receive user inputs useful and/or necessary for controlling the console display 210 and/or the cryo-delivery catheter 300. In some embodiments, the console display 210 could take the form of or include a mobile device and/or wireless device, such as a laptop, desktop computer, tablet, mobile phone, and/or smart television, as examples, and the one or more user interactive mechanism 214 could be user interactive mechanisms of such mobile and/or wireless device.

The cryo-delivery catheter 300 having a thermally controllable distal end probe tip 326 can be delivered through a sheath 110 to the target site, where the tissue to be treated is located. The cryo-delivery catheter 300 can include one or more connectors 320 in the form of wires, cables, tubes, and/or conduits, such as for the exchange of electrical signals, control signals, sensor signals, refrigerant delivery, and/or refrigerant related venting or evacuation. Connectors 320 can couple to the cryogenic console 200 via at least one port 216.

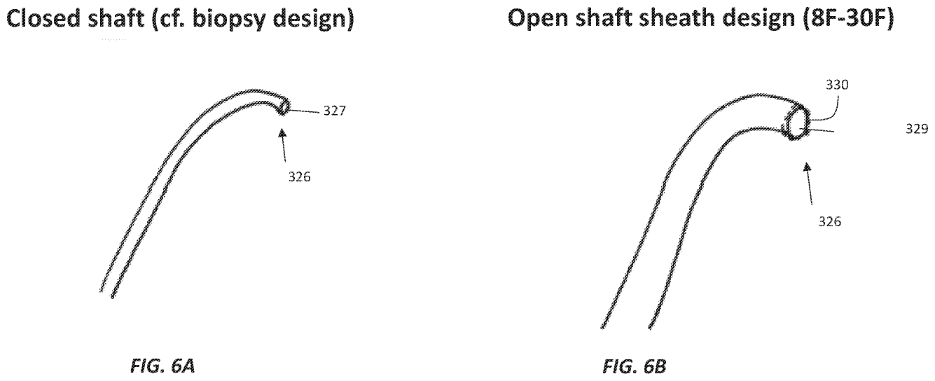

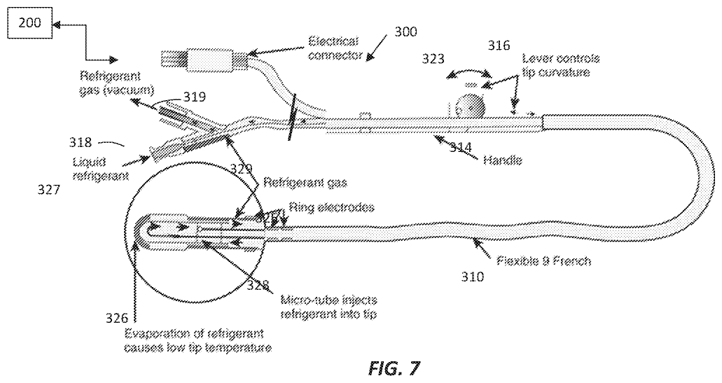

FIG. 6A is an example embodiment of a cryo-delivery catheter 300 with a closed shaft, e.g., closed probe tip 326, in accordance with aspects of the inventive concepts. That is, the probe tip can have a closed end face 327. This embodiment can be used for direct delivery of a therapeutic to tissue. See also FIGS. 7, 8, 9, 10, and 11.

FIG. 6B is an example embodiment of a cryo-delivery catheter 300 with an open shaft, e.g., probe tip 326 with at least one opening, in accordance with aspects of the inventive concepts. That is, the probe tip can have an open end face 329. This embodiment can be used for indirect delivery of a therapeutic to tissue. In this embodiment, the probe tip 326 includes a rim 330 that defines an opening, wherein the rim can be coupled through freezing to the tissue to be treated at the target site.

FIG. 7 is a diagram illustrating an embodiment of a cryo-delivery catheter 300, in accordance with aspects of the inventive concepts. In this embodiment, the cryo-delivery catheter 300 includes at least one elongate, flexible catheter shaft 310 that can be introduced, via sheath 110, into the body of a patient P and, ultimately, into an inner chamber of the heart H. In other embodiments, other organs or tissue could be treated by the cryo-delivery catheter 300, e.g., lungs, liver, stomach, etc.

The cryo-delivery catheter 300 has a proximal end and a distal end. The distal end, which is also the distal end of the catheter shaft 310, comprises the probe tip 326. The proximal end of the cryo-delivery catheter 300 comprises a refrigerant inlet port 318 and a refrigerant outlet port 319. Liquid refrigerant 327 is introduced into the cryo-delivery catheter 300 via the inlet port 318. The liquid refrigerant 327 is injected into the probe tip 326 via at least one micro-tube 328 within the shaft 310 of the cryo-delivery catheter 300. In some embodiments, the catheter shaft 310 comprises a Flexible 9 French tube.

In this embodiment, the probe tip 326 defines a cavity within which the liquid refrigerant evaporates to transition the refrigerant from a liquid state to a gas state, thereby controllably lowering the temperature of the probe tip 326. The thermal controller 240 can regulate the flow of the refrigerant to regulate the temperature of the probe tip. The refrigerant, as gas, is evacuated via a path within the cryo-delivery catheter 300 from the probe tip to the outlet port 319. The outlet port 319 can be a vacuum port configured to connect to a vacuum that aides in the evacuation of the refrigerant gas from the probe tip 326. The pressure of liquid refrigerant drops as it leaves the microtubes 328 and enters the probe tip 326. The decrease in pressure in the probe tip 326 causes the state change to from a liquid to a gas.

At the proximal end of the catheter shaft 310 is a handle 314 that includes lever controls 316 that enable steering of the catheter shaft 310 and probe tip 326 inside the body. The lever controls 316 also enable a user to control and manipulate tip curvature, as seen by arrows 323. The proximal end includes the liquid refrigerant input port 318 and the refrigerant output port 319. The cryo-delivery catheter 300 can include an electrical connector 322 for driving ring electrodes 325 at the distal end and probe tip 326. The electrical connector 322 could be coupled to a properly configured thermal controller 240 that includes cryo-delivery logic for controlling a magnitude and a duration of freezing temperatures at the probe tip 326, and for delivering a therapeutic to a target site of tissue to be treated.

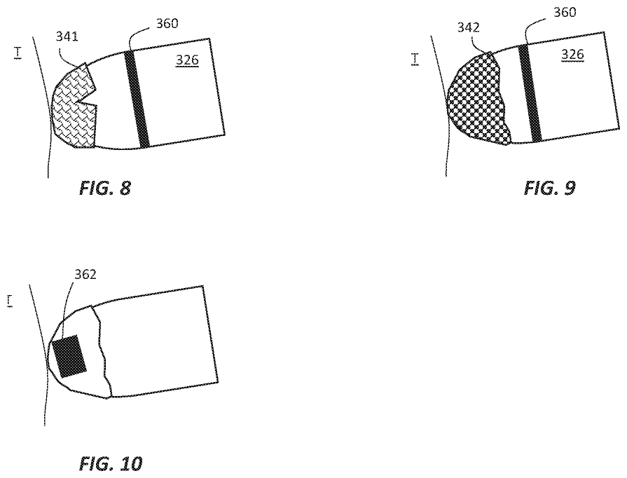

FIG. 8 is an embodiment of a cardiac sheet or patch 341 adhered by freezing to the probe tip 326 at the distal end of the cryo-delivery catheter 300. The cardiac sheet or patch 341 can comprise at least one therapeutic, such as a biologic. FIG. 9 is an embodiment of a material 342 adhered by freezing to the probe tip 326 at the distal end of the cryo-delivery catheter 300. The material 342 can comprise at least one therapeutic, such as a biologic. Once the probe tip 326 is in contact with the tissue T to be treated, the probe tip 326 can be brought above freezing to deliver the therapeutic 341, 342 to the tissue T to be treated. That is, thawing the probe tip can cause the therapeutic on the end of the probe tip to thaw. If the probe tip is in contact with the tissue T at the time of thawing, the therapeutic will also be in contact with the tissue T when the therapeutic thaws, which causes the thawed therapeutic to be delivered and applied to the tissue T to be treated.

As shown in FIGS. 8 and 9, the probe tip 326 can optionally include at least one electrode 360, such as ring electrodes 325. Such electrode 360 could be a cardiac mapping (biopotential electrode) or ultrasound electrode, or a combination thereof. In various embodiments, the electrode 306 could be configured to record electrical potentials and/or to provide localization, which could be in combination with available three-dimensional (3D) mapping systems to track/monitor where material and/or devices have been delivered. In various embodiments, such electrode 360 could be used, therefore, as a part of a guidance system for the probe tip 326 and that material and/or devices delivered therewith.

FIG. 10 shows an embodiment of the probe tip 326 optionally including an electronic device or chip 362. Such electronic device 362 can include a cardiac monitoring or pacing device. As an example, as a pacing device the electronic device 362 could take the form of a leadless pacing device. The electronic device 362 could be embedded in a material delivered to the treatment site tissue T using the cryo-delivery catheter 300. While not shown, at the probe tip 326 could also include least one at least one electrode 360.

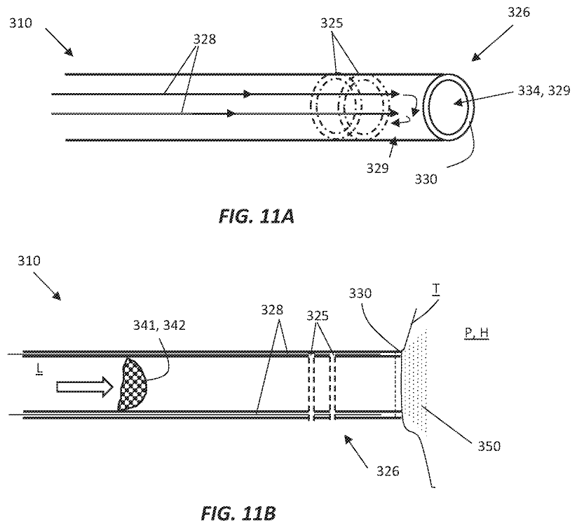

FIG. 11A is an example embodiment of the distal end of the cryo-delivery catheter 300 of FIG. 6B, in accordance with aspects of the inventive concepts. In this embodiment, probe 326 defines at least one opening through which a therapeutic can be delivered. The probe tip 326 includes rim 330 that defines an opening or conduit 334 through which a therapeutic 341, 342 can be delivered to tissue T at the target site. The probe tip 326 can include one or more ring electrodes 325 and one or more microtubes 325 that carry refrigerant to the probe tip 326, as described above. The probe tip 326 and catheter shaft 310 also include one or more paths, tubes, or channels 329 to carry the refrigerant gas away from the probe tip, to be evacuated, as described above.

FIG. 11 is an example embodiment of a cross-section of probe tip 326 of FIG. 11A coupled to tissue T by freezing the rim 330 to the tissue T, at frozen tissue 350. With the rim 330 coupled by freezing to frozen tissue 350, the therapeutic 341, 342 can be delivered through at least one lumen L in the catheter shaft 310. After delivery, the probe tip 326 can be brought above freezing to release the rim 330 from the tissue T.

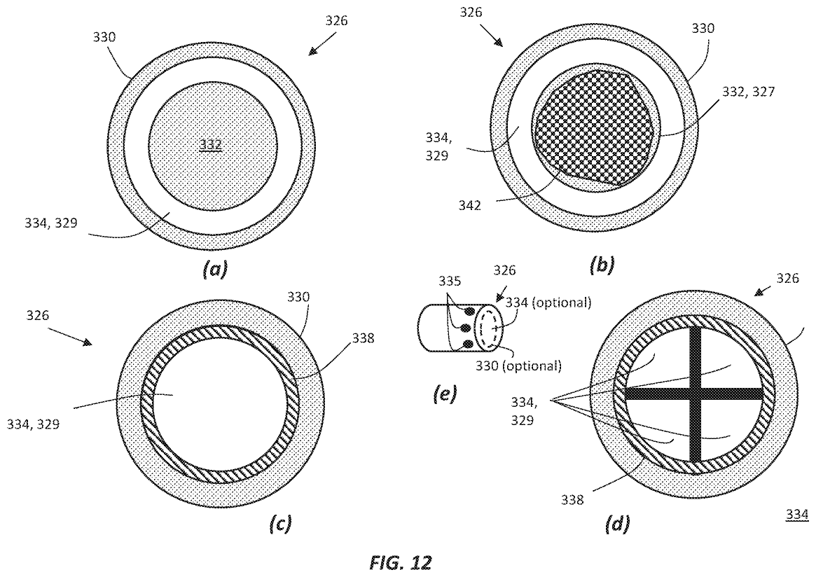

FIG. 12(a)-(e) are example embodiments of different configurations of a distal end of a cryo-delivery catheter, in accordance with aspects of the inventive concepts.

FIG. 12(a)-(b) show an embodiment of a probe tip 326 having a core 332 that can be temperature controlled and a ring 330 that can be temperature controlled. In this embodiment, the probe tip can support both methods of delivery, direct and indirect. An opening or conduit 334 exists between core 332 and ring 330 through which a therapeutic can be delivered. And, in some embodiments, core 332 can also be used for direct delivery of a therapeutic by freezing a therapeutic to the core.

FIG. 12(c) is another example embodiment of a configuration of a probe tip 326 having an open end. In this embodiment, the probe tip 326 includes the ring 330, but the ring, at least at the distal end, is lined with a thermal insulator 338. The insulator 338 can maintain the opening above freezing while the ring is at or below freezing. As a result, the therapeutic 341, 342 may bass through the opening 334 more freely, without increased rigidity or reduced viscosity caused by the freezing temperature of the ring 330.

FIG. 12(d) is another example embodiment of a configuration of a probe tip 326 having an open end. In this embodiment, the probe tip 326 includes the ring 330, but the ring, at least at the distal end, is lined with a thermal insulator 338. The insulator 338 can maintain the opening above freezing while the ring is at or below freezing. The probe tip 326, and the catheter shaft 310, may include a plurality of conduits 334 for delivery of a plurality of different agents therethrough, while the ring is frozen to the tissue T. As examples, the different agents can include an antibiotic, one or more biologics, therapeutics, dyes, anti-inflammatoires, and so on. As a result, the one or more agents 341, 342 may pass through the opening 334 more freely, without increased rigidity o reduced viscosity caused by the freezing temperature of the ring 330.

In other embodiments, the probe tip 326 can include multiple conduits 334 without the insulator or one or more of the conduits can be lined with an insulator.

FIG. 12(e) is another example embodiment of a configuration of a probe tip 326. In this embodiment, the probe tip includes one or more openings 334 formed in a side of the probe tip 326. With this embodiment, the end can be closed and can be temperature controlled to adhere by freezing to the tissue T at the target site. With the probe tip 326 adhered to the tissue through freezing, a therapeutic can be delivered through the side openings 334. In other embodiments, the probe tip may include a temperature controlled ring 330 that can frozen to the tissue for delivery of a therapeutic. In such case, a therapeutic could be delivered via opening defined by the opening within the ring 330 and through the side openings 335.

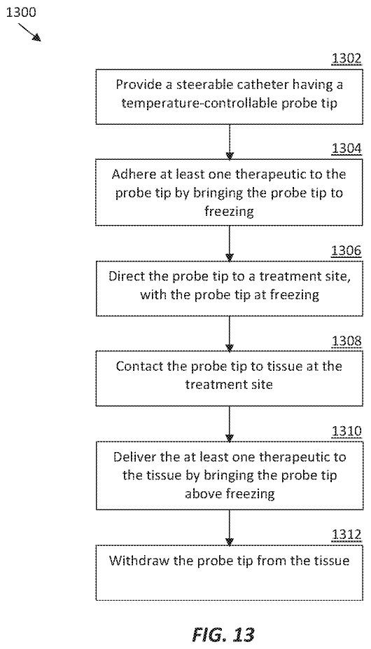

FIG. 13 is a flowchart depicting an embodiment of a method of percutaneous delivery of a treatment agent 1300, in accordance with aspects of the inventive concepts. The percutaneous cryo-delivery method 1300 can comprise providing a steerable catheter having a probe tip having a probe tip temperature that is controllable by a thermal controller (1302); adhering at least one therapeutic to the probe tip outside the body by using the thermal controller to maintain the probe tip temperature at or below freezing while in contact with the at least one therapeutic (1304); using the steerable catheter, directing the probe tip with the adhered at least one therapeutic to a treatment site within the body while maintaining the probe tip at or below freezing (1306, 1308); and using the thermal controller, delivering the at least one therapeutic to the treatment site by causing the probe tip temperature to rise above freezing while in contact with the treatment site (1310). Thereafter, the probe tip can be withdrawn from the tissue site, and the body (1312).

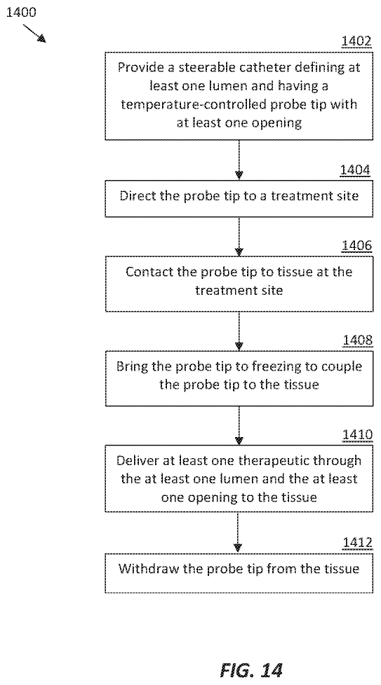

FIG. 14 is a flowchart depicting another embodiment of a method of percutaneous delivery of a treatment agent 1400, in accordance with aspects of the inventive concepts. The percutaneous cryo-delivery method 1400 can comprise providing a steerable catheter defining at least one lumen and having a probe tip having at least one opening, where a probe tip temperature is controllable by a thermal controller (1402); delivering the probe tip to contact a treatment site within the body (1404, 1406); using the thermal controller, adhering the probe tip to the treatment site by bringing the probe tip temperature to or below freezing (1408); and, while maintaining the probe tip frozen adherence to the treatment site, delivering at least one therapeutic to the treatment site through the at least one lumen and the at least one opening of the probe tip (1410). Thereafter, the probe tip can be withdrawn from the tissue site, and the body (1412).

In various embodiments, the cryo-delivery catheter can be configured to use electroporation and/or other approaches to facilitate transfer of material into the cardiac tissue. “Electroporation, or electropermeabilization, is a microbiology technique in which an electrical field is applied to cells in order to increase the permeability of the cell membrane, allowing chemicals, drugs, electrode arrays or DNA to be introduced into the cell (also called electrotransfer).” (See Wikipedia, at https://en.wikipedia.org/wiki/Electroporation.) Such a technique can be used to increase the permeability of tissue at the site that will receive a therapeutic via the cryo-delivery apparatus and method.

In various embodiments, the cryo-delivery apparatus can be used to deliver and/or place devices acting as biological compound/drug reservoirs for slow-release or time-release therapeutics at a treatment site.

In various embodiments, the cryo-delivery apparatus can be used to deliver and/or place plastic bioelectronics as a form of therapeutics at a treatment site. Plastic bioelectronics takes advantage of the inherent properties of polymers and soft organic electronics for applications at the interface of biology and electronics. The resulting electronic materials and devices are soft, stretchable and mechanically conformable, making them suitable for interacting with biological systems in implantable devices. Such devices can, therefore, be suitable as implantable devices delivered via, e.g., through or by, the cryo-delivery apparatus.

It will be appreciated by those skilled in the art that certain features of the invention or inventive concepts, which are, for clarity, described in the context of separate embodiments, may also be provided in combination in a single embodiment. Conversely, various features of the invention which are, for brevity, described in the context of a single embodiment may also be provided separately or in any suitable sub-combination.

Below follows an itemized list of statements describing embodiments in accordance with the inventive concepts:
1. A percutaneous cryo-delivery method, comprising:
   providing a steerable catheter having a probe tip having a probe tip temperature that is controllable by a thermal controller;
   adhering a therapeutic to the probe tip outside the body by using the thermal controller to maintain the probe tip temperature at or below freezing while in contact with the therapeutic;
   using the steerable catheter, directing the probe tip with the adhered therapeutic to a treatment site within the body while maintaining the probe tip at or below freezing; and
   using the thermal controller, delivering the therapeutic to the treatment site by causing the probe tip temperature to rise above freezing while in contact with the treatment site.
2. The method of statement 1, wherein the catheter defines at least one lumen and the probe tip defines at least one opening terminating the at least one lumen and the method includes delivering at least one therapeutic to the treatment site via the at least one lumen and the at least one opening.
3. A percutaneous cryo-delivery method, comprising:
   providing a steerable catheter defining at least one lumen and having a probe tip having at least one opening, where a probe tip temperature is controllable by a thermal controller;
   delivering the probe tip to contact a treatment site within the body;
   using the thermal controller, adhering the probe tip to tissue at the treatment site by bringing the probe tip temperature to or below freezing; and
   while maintaining the probe tip frozen adherence to the tissue, delivering a therapeutic to the tissue through the at least one lumen and the at least one probe tip opening.
4. A percutaneous cryo-delivery method, comprising:
   providing a steerable catheter defining a lumen and having a probe tip including a rim, where a rim temperature is controllable by a thermal controller;
   delivering the rim to contact tissue at a treatment site within the body;
   using the thermal controller, adhering the rim to the tissue by bringing the rim temperature to or below freezing; and
   while maintaining the rim in frozen adherence with the tissue, delivering a therapeutic to the tissue through the lumen and via the rim.
5. The method of any of statements 1 through 4, wherein the therapeutic is in the form of a material and/or at least one device.
6. The method of any of statements 1 through 4, wherein the therapeutic is in the form therapeutic a drug, biologic, material, and/or device having a treatment and/or ameliorative effect, which is delivered via the cryo-delivery catheter to a site and/or tissue within the body.
7. The method of any of statements 1 through 5, wherein the therapeutic includes a monitoring device, a pacing device, and/or a leadless pacing device.
8. The method of any of statements 1 through 4, wherein the therapeutic is a biologic.
9. The method of any of statements 1 through 7, wherein the therapeutic includes plastic bioelectronics.
10. The method of any of statements 1 through 4, 6 and 8, wherein the therapeutic includes one or more of the following: RNA, DNA, stem cells, progenitor cells, tissue, drugs, and/or CRISPR-Cas.
11. The method of any of statements 1 through 7, wherein the therapeutic is a delivered in a format of a patch or sheet.
12. The method of any of statements 1 through 4 wherein the therapeutic is in the form of a gel, paste, liquid, solution, solid, or combinations thereof.
13. The method of any of statements 1 through 12, wherein the probe tip includes at least one opening and the method includes delivering at least one therapeutic through the at least one opening.
14. The method of any of statements 1 through 13, wherein the probe tip includes a plurality of openings and the method includes delivering a single therapeutic through the plurality of openings.
15. The method of any of statements 1 through 4, wherein the probe tip includes a plurality of openings and the method includes delivering a plurality of therapeutics through the plurality of openings.
16. The method of any of statements 1 to 15, wherein the probe tip includes at least one thermal insulator insulting the at least one opening and/or rim from the lumen or plurality of lumens and the therapeutic or plurality of therapeutics.
17. The method of any of statements 1 to 16, wherein the probe includes an electronic device including at least one electrode, such as a biopotential electrode and/or an ultrasound electrode.
18. The method of statement 17, wherein the at least one electrode can comprise at least one ring electrode.
19. The method of any of statements 1 through 4, wherein delivering the therapeutic includes delivering a monitoring device, a pacing device, and/or a leadless pacing device to the treatment site.
20. The method of any of statements 1 to 19, further comprising using electroporation or electropermeabilization to increase a permeability of the tissue at the treatment site.
21. A cryo-delivery catheter, comprising:
   an elongate shaft defining at least one lumen;
   a probe tip coupled to a distal end of the shaft and having at least one opening terminating the at least one lumen; and
   a thermal controller configured to control a temperature of the probe tip to bring a probe tip temperature to or below freezing.
22. The catheter of statement 21, wherein the at least one opening includes at least one rim configured to achieve the probe tip temperature at or below freezing and configured to couple by freezing to tissue at a treatment site.
23. The catheter of statements 21 or 22, wherein the probe tip includes one opening defined by one rim.
24. The catheter of statements 21 through 23, wherein the probe tip includes a plurality of openings within one rim.
25. The catheter of statement 21, wherein the probe tip includes a plurality of openings defined by a plurality of rims.
26. The catheter of statements 21 or 25, wherein one or more of the plurality of rims is independently thermally controllable to reach a temperature at or below freezing.
27. The catheter of statement 21, wherein the probe tip includes at least one insulator insulating the at least one opening, the at least one lumen, and/or the rim or plurality of rims.
28. The catheter of statements 21-23, 26, or 27, wherein the probe tip includes one opening or rim and an insulator insulating the one opening or rim.
29. The catheter of any of statements 21 to 28, further comprising at least one microtube configured to carry a refrigerant to at least one void in the probe tip and at least one conduit configured to evacuate refrigerant gas from the at least one void.
30. The catheter of any of statements 21 to 29, or any other statement or combinations of statements, wherein the at least one lumen is configured as a pathway for at least one therapeutic to exit the probe tip via the at least one opening, rim, or plurality of rims while the at least one opening is frozen to the tissue at the treatment site.
31. The catheter of any of statements 21 to 30, the probe tip further includes a temperature controllable end face that encloses at least a portion of the probe tip and is configured to maintain a therapeutic by freezing.
32. The catheter of statement 31, wherein the probe tip end face is temperature controllable to transition above freezing to deliver the therapeutic to the tissue at the treatment site.
33. The catheter of statements 21 through 32, wherein the probe includes an electronic device including at least one electrode, such as a biopotential electrode and/or an ultrasound electrode.
34. The catheter of statement 33, wherein the at least one electrode can comprise at least one ring electrode.
35. The catheter of statements 21 through 34, wherein the catheter is configured to deliver a therapeutic in the form of a material and/or at least one device.
36. The catheter of statements 21 through 35, wherein the catheter is configured to deliver a therapeutic to a site and/or tissue within the body in the form of a drug, biologic, material, and/or device having a treatment and/or ameliorative effect.
37. The catheter of statement 21 through 36, wherein the catheter is configured to deliver a therapeutic to a site and/or tissue within the body that includes a monitoring device, a pacing device, and/or a leadless pacing device.
38. The catheter of statements 21 through 37, wherein the catheter is configured to deliver a therapeutic to a site and/or tissue within the body that includes a biologic.
39. The catheter of statements 21 through 38, wherein the catheter is configured to deliver a therapeutic to a site and/or tissue within the body that includes plastic bioelectronics.
40. A cryo-delivery probe:
configured to couple to a distal end of a catheter shaft having at least one lumen;
at least one opening configured to terminate the at least one lumen; and
a temperature controllable tip configured to transition to a probe tip temperature at or below freezing to couple by freezing to tissue of a treatment site.
41. The probe of statement 40, or any other statement or combinations of statements, wherein the at least one opening includes at least one rim configured to achieve the probe tip temperature at or below freezing and configured to couple by freezing to tissue at a treatment site.
42. The probe of statement 40, or any other statement or combinations of statements, wherein the probe tip includes one opening defined by one rim.
43. The probe of statement 40, or any other statement or combinations of statements, wherein the probe tip includes a plurality of openings within one rim,
44. The probe of statement 40, or any other statement or combinations of statements, wherein the probe tip includes a plurality of openings defined by a plurality of rims.
45. The probe of statement 40, or any other statement or combinations of statements, wherein one or more of the plurality of rims is independently thermally controllable to reach a temperature at or below freezing.
46. The probe of statement 40, or any other statement or combinations of statements, wherein the probe tip includes at least one insulator insulating the at least one opening, the at least one lumen, and/or the rim or plurality of rims.
47. The probe of statement 40, or any other statement or combinations of statements, wherein the probe tip includes one opening or rim and an insulator insulating the one opening or rim.
48. The probe of statement 40, or any other statement or combinations of statements, wherein the probe tip further includes a temperature controllable end face the closes at least a portion of the probe tip and is configured to maintain a therapeutic by freezing.
49. The probe of statement 48, or any other statement or combinations of statements, wherein the probe tip end face is temperature controllable to transition above freezing to deliver the therapeutic to the tissue at the treatment site.
50. The probe of statement 40, or any other statement or combinations of statements, wherein the probe tip includes an electronic device including at least one electrode, such as a biopotential electrode and/or an ultrasound electrode.
51. The probe of statement 50, or any other statement or combinations of statements, wherein the at least one electrode can comprise at least one ring electrode.
52. The probe of statement 40, or any other statement or combinations of statements, wherein the probe tip is configured to deliver a therapeutic in the form of a material and/or at least one device.
53. The probe of statement 40, or any other statement or combinations of statements, wherein the probe tip is configured to deliver a therapeutic to a site and/or tissue within the body in the form of a drug, biologic, material, and/or device having a treatment and/or ameliorative effect.
54. The probe of statement 40, or any other statement or combinations of statements, wherein the probe tip is configured to deliver a therapeutic to a site and/or tissue within the body that includes a monitoring device, a pacing device, and/or a leadless pacing device.
55. The probe of statement 40, or any other statement or combinations of statements, wherein the probe tip is configured to deliver a therapeutic to a site and/or tissue within the body that includes a biologic.
56. The probe of statement 40, or any other statement or combinations of statements, wherein the probe tip is configured to deliver a therapeutic to a site and/or tissue within the body that includes plastic bioelectronics.

While the foregoing has described what are considered to be the best mode and/or other preferred embodiments, it is understood that various modifications can be made therein and that the invention or inventions may be implemented in various forms and embodiments, and that they may be applied in numerous applications, only some of which have been described herein. It is intended by the following claims to claim that which is literally described and all equivalents thereto, including all modifications and variations that fall within the scope of each claim.

It is appreciated that certain features of the invention, which are, for clarity, described in the context of separate embodiments, may also be provided in combination in a single embodiment. Conversely, various features of the invention which are, for brevity, described in the context of a single embodiment may also be provided separately or in any suitable sub-combination.

It will be appreciated that all of the features set out in any of the claims (whether independent or dependent) can combined in any given way.

1. A percutaneous cryo-delivery method, comprising:
providing a steerable catheter having a probe tip having a probe tip temperature that is controllable by a thermal controller;
adhering a therapeutic to the probe tip outside the body by using the thermal controller to maintain the probe tip temperature at or below freezing while in contact with the therapeutic;
using the steerable catheter, directing the probe tip with the adhered therapeutic to a treatment site within the body while maintaining the probe tip at or below freezing; and
using the thermal controller, delivering the therapeutic to the treatment site by causing the probe tip temperature to rise above freezing while in contact with the treatment site.

2.-56. (canceled)
